(19) United States
(12) Patent Application Publication
(10) Pub. No.: US 2026/0315454 A1
(43) Pub. Date: Oct. 15, 2026

(54) Use of quinoline-4-carboxylic acid in the preparation of anti-aging product as well as product for protecting liver and promoting liver regeneration

(71) Applicant: Guangzhou Riju Trade Development Co., Ltd., Guangzhou (CN)

(72) Inventors: Jingxing Ou, Guangzhou (CN); Jinghong Yang, Guangzhou (CN); Xiaomei Zhang, Guangzhou (CN); Weizhao Chen, Guangzhou (CN); Jingyun Ou, Guangzhou (CN); Wei Liu, Guangzhou (CN)

(21) Appl. No.: 18/728,010

(22) PCT Filed: Nov. 23, 2023
(86) PCT No.: PCT/CN2023/133623

§ 371 Date: Jul. 10, 2024

(30) Foreign Application Priority Data
Nov. 14, 2023 (CN) 202311520155.3

(51) Int. Cl. A61K 31/47; A61P 1/16; A61P 21/00; A61P 39/06; A61P 43/00

(52) U.S. Cl. CPC A61K 31/47; A61P 1/16; A61P 21/00; A61P 39/06; A61P 43/00

(57) ABSTRACT

The present disclosure provides use of quinoline-4-carboxylic acid in the preparation of an anti-aging product as well as a product for protecting liver and promoting liver regeneration, belonging to the field of pharmaceutical technology. The present disclosure discovers that: quinoline-4-carboxylic acid can be used as a novel additive for the in vitro expansion culture of mesenchymal stem cells, breaking through the bottleneck that mesenchymal stem cells cultured in vitro are susceptible to aging, and optimizing their clinical development and application. Quinoline-4-carboxylic acid can alleviate the aging-related phenotypes of fruit flies and mice, enhance the exercise capacity of fruit flies and the muscle strength of mice, prolong the lifespan of fruit flies and mice, and is expected to be an effective component of anti-aging treatment drugs or healthcare products. Quinoline-4-carboxylic acid can improve the survival rate of mice after hepatectomy, promote liver regeneration after hepatectomy injury, and is expected to be an active ingredient in drugs or healthcare products to promote the prognosis of patients after hepatectomy, to promote liver regeneration, and to alleviate or treat liver injury.

### TECHNICAL FIELD

The present disclosure belongs to the field of pharmaceutical technology, and particularly relates to use of quinoline-4-carboxylic acid in the preparation of an anti-aging product as well as a product for protecting liver and promoting liver regeneration.

### BACKGROUND

Aging is characterized by a progressive loss of physiological integrity, leading to impaired function and increased risk of death. For decades, biologists have been faced with the challenge of retarding aging and prolonging lifespan. Most hibernating animals live longer than their non-hibernating counterparts, which exhibit excellent metabolic adaptation and damage protection in extreme environments. Thus hibernating animal models are excellent models for studying metabolism, hypoxia/reperfusion and longevity. As a hibernating animal, the thirteen-lined ground squirrel tolerates low temperatures and adapts to cold by reducing cold sensitivity in the peripheral somatosensory system and preventing reactive oxygen species-induced cellular damage. Similarly, the induced pluripotent stem cells of the thirteen-lined ground squirrel (GS iPSC) show a higher adaptability to stress environments. The GS iPSC cell model is used to investigate the protective mechanism of cold adaptation in hibernating animals from a metabolic perspective, with a view to applying the mechanism to the field of anti-aging to address human aging-related diseases.

Quinoline-4-carboxylic acid (QCA) is a quinoline monocarboxylic acid identified from the metabolomics analysis by freezing and rewarming of GS iPSC cells. The available data suggest that QCA belongs to quinoline derivatives, which may be potential downstream products of tryptophan metabolism. Quinoline derivatives are of interest for their broad-spectrum antimalarial, anticancer, antibacterial, antimicrobial, antifungal and other effects. In addition, many studies have suggested that quinoline derivatives have protective effects against oxidative stress and attenuate age-related oxidative damage in rats. However, due to the limited data available in the literature, it is unclear whether QCA exerts a protective effect in aging and regeneration models.

Mesenchymal stem cells (MSCs) are mesoderm-derived pluripotent stem cells with multidirectional differentiation potential, and are important members of the stem cell family. Currently, bone marrow-derived and umbilical cord-derived mesenchymal stem cells are more frequently studied. Because of their multidirectional differentiation ability and multiple cytokine secretion function, MSCs have been widely used in clinical applications, mainly for neurological injuries, liver diseases, myocardial ischemia, diabetes, and skin problems. The most widely used stem cell product on the market is a mesenchymal stem cell. With the deepening of research, scientists have found that MSCs have characteristics such as promotion of stem cell implantation, hematopoietic support, immune regulation and self-replication in addition to their multidirectional differentiation potential, and have gradually become one of the hotspots for research in this field. Usually, the number of cells isolated from donors is limited, and in order to obtain a sufficient amount of cells for clinical treatment, it is inevitable that MSCs are expanded and cultured in vitro. The MSCs cultured in vitro for a long period of time are susceptible to aging, which is a constraint to their clinical development and application. Thus, it is of great significance to explore the main mechanisms of MSC aging and how to alleviate MSC aging, and to develop safe and effective anti-aging additives for in vitro culture.

As the center of material metabolism in the human body, the liver plays an important role in a variety of physiological and pathological processes, and many diseases also involve the liver. Partial hepatectomy is currently the most common and effective treatment method for liver tumors, and the strong regeneration ability of normal liver tissue is the basis for effective hepatectomy. However, limited by the bottleneck of early diagnostic techniques for liver tumors, most liver tumors have already progressed to intermediate and advanced stages when they are first diagnosed. Due to the large volume of the tumor and the large number of liver segments involved, the volume of the residual liver after resection is unable to meet the metabolic demands of the human body, which can lead to life-threatening complications such as liver failure. In order to solve the problem of insufficient residual liver volume, several surgical methods have been developed clinically to stimulate rapid regeneration of residual liver, including Portal Vein Ligation (PVL)/Portal Vein Embolization (PVE), and Associating Liver Partition and Portal Vein Ligation for Staged Hepatectomy (ALPPS). However, the results of these procedures are controversial, and the risk of liver failure in postoperative patients remains high due to insufficient residual liver volume. Therefore, developing safe and effective drugs for promoting liver regeneration, accelerating the regeneration of residual liver in the perioperative period, and reducing the occurrence rate of liver failure are of great significance for the surgical treatment of liver cancer.

Aging is accompanied by a significant increase in a variety of diseases, and how to retard aging has been a hotspot of research among scientists. Stem cells play an important role in the aging process, and the aging of stem cells accelerates the aging of the human body. Mesenchymal stem cells (MSCs) are a kind of stem cells with self-replicating ability and multidirectional differentiation potential. Although the existing in vitro expansion media of MSCs can enable same to be expanded and cultured in vitro for several generations, they still fail to achieve a satisfactory effect in terms of retarding cell aging. Also, as mentioned above, the effect of the existing surgery to stimulate rapid regeneration of residual liver is still controversial, and the risk of liver failure in postoperative patients remains high due to insufficient residual liver volume. Therefore, further research and development of novel technical solutions or drugs to retard aging (stem cell aging) and promote liver regeneration after hepatectomy are needed to lay the foundation for clinical application.

### SUMMARY

In order to solve the above technical problems, the present disclosure provides use of quinoline-4-carboxylic acid in the preparation of an anti-aging product as well as a product for protecting liver and promoting liver regeneration. Specifically, quinoline-4-carboxylic acid can be used as a novel additive for the in vitro expansion culture of mesenchymal stem cells, breaking through the bottleneck that mesenchymal stem cells cultured in vitro are susceptible to aging, and optimizing their clinical development and application. Quinoline-4-carboxylic acid can alleviate the aging-related phenotypes of fruit flies and mice, enhance the exercise capacity of fruit flies and the muscle strength of mice, prolong the lifespan of fruit flies and mice, and is expected to be an effective component of anti-aging treatment drugs or healthcare products; Quinoline-4-carboxylic acid can improve the survival rate of mice after hepatectomy, promote liver regeneration after hepatectomy injury, and is expected to be an active ingredient in drugs or healthcare products to promote the prognosis of patients after hepatectomy, to promote liver regeneration, and to alleviate or treat liver injury.

In order to realize the above objects, the present disclosure employs the following technical solutions:

One object according to the present disclosure is to provide use of quinoline-4-carboxylic acid in the preparation of an anti-aging product.

Further, the anti-aging product is specifically a product for retarding the aging of mesenchymal stem cells.

Still further, the product for retarding the aging of mesenchymal stem cells is specifically a product for promoting the proliferation of mesenchymal stem cells and/or a product for reducing the nuclear DNA damage of mesenchymal stem cells.

Further, the anti-aging product is specifically a product for enhancing exercise capacity and muscle strength.

A second object according to the present disclosure is to provide use of quinoline-4-carboxylic acid in the preparation of a product for protecting liver.

A third object according to the present disclosure is to provide use of quinoline-4-carboxylic acid in the preparation of a product for promoting liver regeneration.

A fourth object according to the present disclosure is to provide use of quinoline-4-carboxylic acid in the preparation of a product for improving the survival rate after hepatectomy.

Further, the product for protecting liver, the product for promoting liver regeneration, and the product for improving the survival rate after hepatectomy are specifically a product for reducing liver inflammation and/or a product for reducing liver injury.

Compared with the prior art, the present disclosure has the following technical effects:

Taking hibernating animals as a natural model, the present inventor innovatively uses induced pluripotent stem cells of hibernating animals, thirteen-lined ground squirrels, to explore the molecular mechanism and its metabolic regulation mechanism during cold adaptation and rewarming of hibernating animals, and discovers that quinoline-4-carboxylic acid increases during cold adaptation and decreases during rewarming, locking in the fact that it may be a metabolite that can slow down the damage caused by stress. It is expected to be applied to resist aging-related damage, retard aging and promote regeneration and repairing. And specific tests found that: the addition of quinoline-4-carboxylic acid to the medium of mesenchymal stem cells cultured in vitro can promote the proliferation of aging mesenchymal stem cells, reduce the nuclear DNA breakage, and retard their aging; quinoline-4-carboxylic acid can alleviate the symptoms such as poor coat and dermatitis in old mice; quinoline-4-carboxylic acid can down-regulate the enzymatic indexes of chronic inflammation and liver injury; quinoline-4-carboxylic acid can enhance the arm strength of old mice and prolong the lifespan of mice; quinoline-4-carboxylic acid can enhance the exercise capacity of fruit flies and prolong the lifespan of fruit flies; quinoline-4-carboxylic acid can promote the regeneration ability of liver after hepatectomy in old mice, improve the survival rate after hepatectomy in old mice and attenuate the enzymatic indexes of liver injury, etc.

### BRIEF DESCRIPTION OF FIGURES

FIG. 1 is a graph showing nuclear DNA breakage in GS iPSC and hMSC cells after the freezing-rewarming process in Example 1 according to the present disclosure;

FIG. 2 is volcano plot and violin plot showing the quinoline-4-carboxylic acid in the freezing-rewarming metabolome of GS iPSC cells in Example 1 according to the present disclosure;

FIG. 3 is a violin plot showing the content changes of quinoline-4-carboxylic acid in cold storage-normothermic perfusion metabolome of hamster liver in Example 1 according to the present disclosure;

FIG. 4 is a graph showing the results of the effect of adding different concentrations of quinoline-4-carboxylic acid to the medium of high-generation mesenchymal stem cells on the proliferation of cells in Example 1 according to the present disclosure;

FIG. 5 is a graph showing the nuclear DNA breakage of high-generation mesenchymal stem cells and the reduction of aging cells after the treatment with quinoline-4-carboxylic acid in Example 1 according to the present disclosure;

FIG. 6 is a graph showing the survival curves of fruit flies and the results of tube-climbing tests after the addition of quinoline-4-carboxylic acid in Example 1 according to the present disclosure;

FIG. 7 is a graph showing the changes in coat, dermatitis, arm strength, chronic inflammation and enzymatic indexes of liver injury in old mice after the addition of quinoline-4-carboxylic acid in Example 1 according to the present disclosure; and

FIG. 8 is a graph showing the effects of the addition of quinoline-4-carboxylic acid on regenerative liver to body weight ratio after hepatectomy, survival rate after hepatectomy, and enzymatic indexes of liver injury in mice in Example 1 according to the present disclosure.

### DETAILED DESCRIPTION

The following examples are used to illustrate the present disclosure, but are not intended to limit the scope of the disclosure. Without departing from the spirit and essence of the present disclosure, modifications or substitutions made to the methods, steps or conditions according to the present disclosure are within the scope of the present disclosure. The equipment and raw materials and the like as used in the following examples are commercially available, and the methods as used in the examples are consistent with those conventionally used if not otherwise specified.

The technical solutions according to the present disclosure are described in further detail below in connection with the examples.

### Example 1

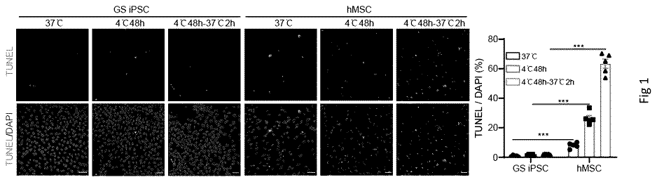

1. GS iPSC or hMSC cultured under normal conditions was used as a sample in 37° C. control group. For cold-exposure treatment, the medium was replaced with a UW solution. After being placed at room temperature for 10 min, the cells in UW solution were transferred to a refrigerator at 4° C. and stored for 48 h. The rewarming treatment was performed by removing the cells from the refrigerator and replacing the medium with a pre-cooled medium. After being placed at room temperature for 10 min, the cells were transferred to an incubator at 37° C. for 2 h and then the samples at 4° C. for 4 h and 37° C. for 2 h were collected. Cells were fixed by 4% paraformaldehyde and then subjected to a TUNEL staining test. The staining results were shown in FIG. 1.

The results showed that after subjecting the cells to freeze-rewarming, the hMSC shows an increased number of nuclear DNA breakage compared to GS iPSC in TUNEL staining, suggesting that GS iPSC have a higher adaptability to stress environments.

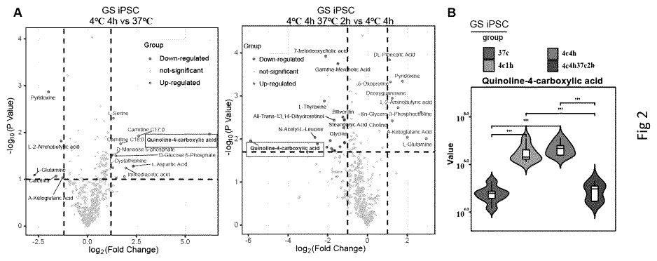

2. GS iPSC cultured under normal conditions was used as a sample in 37° C. control group. For the cold-exposure treatment, the GS iPSC medium was replaced with a Hibernate-A medium. After being placed at room temperature for 10 min, the cells in Hibernate-A medium were transferred to a refrigerator at 4° C. and stored for 4 h, and the samples at 4° C. for 4 h were collected. The rewarming treatment was performed by removing the cells from the refrigerator and replacing the medium with a pre-cooled GS iPSC medium. After being placed at room temperature for 10 min, the cells were transferred to an incubator at 37° C. for 2 h and then the samples at 4° C. for 4 h and 37° C. for 2 h were collected. The samples collected were sent to the company for metabolomics. By analyzing the metabolomics data, the volcano plot and violin plot were made using the website of Bioinformatics Graphics, and the results were shown in FIG. 2.

Both the volcano plot and violin plot showed that quinoline-4-carboxylic acid increases during cold adaptation and decreases during rewarming.

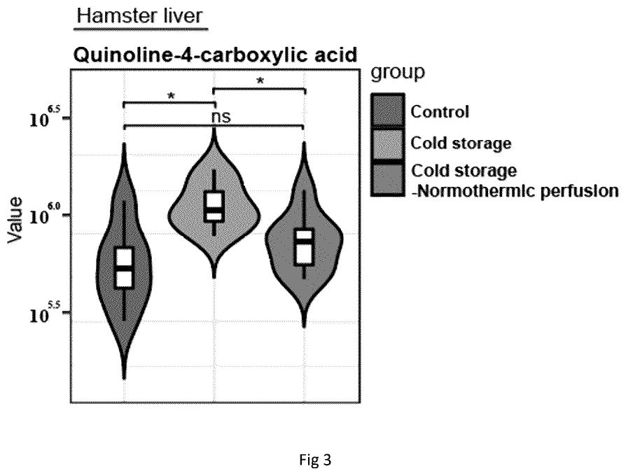

3. After an overnight fasting period, 2-3 month-old male/female SD rats or 2-3 month-old golden hamsters were anesthetized by inhalation of isoflurane and given 50 IU heparin. A PE-10 catheter was used for bile duct intubation and a 22-G Introcan catheter was used for portal vein intubation. The liver was then flushed with saline and UW (University of Wisconsin, UW) solution, at which time the liver was collected as a liver sample in control group. A perfusion system was established using a recirculating approach, and 250 ml of Krebs-Henseleit (KH) bicarbonate buffer was prepared and oxygenated through a fiber oxygenator in a mixture of 95% O₂ and 5% CO₂ to achieve a partial pressure of oxygen of over 500 mmHg. Liver samples from the cold storage group were collected after cold storage at 4° C. for 48 h. The samples were equilibrated at room temperature for 10 min, and perfused at 37° C. for 2 h. After which, the normothermic perfusion samples after cold storage were collected. The flow was set at pressure-controlled mode, and the portal vein pressure (PVP) was kept at 12 mmHg. The flow and portal vein pressure were automatically monitored and recorded. Portal vein resistance (PVR) was calculated as: PVR (mmHg/mL×min×g liver)=PVP (12 mmHg)/portal vein flow (mL×min×g liver). The samples collected were sent to the company for metabolomics. By analyzing the metabolomics data, a violin plot was made using the website of Bioinformatics Graphics, and the results were shown in FIG. 3.

The violin plot showed that quinoline-4-carboxylic acid increases during cold storage and decreases during normothermic perfusion in hamster liver.

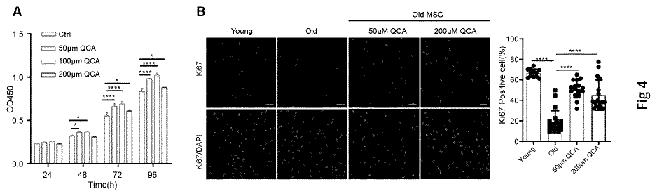

4. High-generation MSCs (Old MSCs) at P10-15 were plated in a 96 plate, and 50 μM, 100 μM, and 200 μM of QCA were added to the medium, respectively. The medium without adding QCA was used as a control. The CCK8 assay was performed, and the results were shown in FIG. 4A. Low-generation MSCs (Young MSCs) at P4-7, high-generation MSCs (Old MSCs) at P10-15, as well as high-generation MSCs respectively treated with 50 μM and 200 μM QCA for 72 h, were subjected to Ki67 immunofluorescence staining, and the results were shown in FIG. 4B.

CCK8 assay and Ki67 immunofluorescence staining showed that the addition of quinoline-4-carboxylic acid to the medium of high-generation MSC can promote cell proliferation.

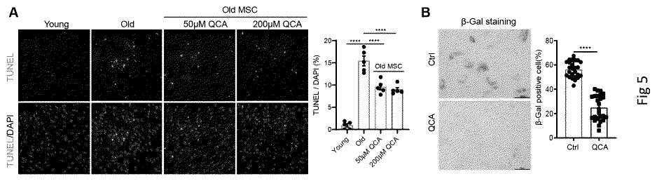

5. Low-generation MSCs (Young MSCs) at P4-7, high-generation MSCs (Old MSCs) at P10-15, as well as old MSCs respectively treated with 50 μM and 200 μM QCA for 72 h were subjected to TUNEL staining, and the results were shown in FIG. 5A. Old MSCs with 50 μM QCA were added to the medium all the time (a fresh medium containing QCA was changed every 2-3 days), and the medium without adding QCA was used as a control. The cells were passaged for 3 consecutive generations (passaged when the cells were 80-90% confluent) before being used for β-Gal staining tests, and the results were shown in FIG. 5B.

TUNEL staining and β-Gal staining showed that the addition of quinoline-4-carboxylic acid to the Old MSC medium reduces nuclear DNA breakage and retards cell aging.

(6) The regular medium for rearing fruit flies was added with a preservative and dispensed. After which, 115 μL of QCA (100 mM, equivalent to 2 mg QCA/tube) was added dropwise to the medium, and the same volume of 95% ethanol was added to the control tube. On days 20-30 and 40-50 of fruit flies rearing, the tubes were replaced with QCA-containing rearing tubes for rearing. 3 male W1118 fruit flies and 3 virgin W1118 fruit flies were added to each rearing tube and bred for 3-5 days for a total of 20 tubes. The parental fruit flies were cleared after about 5-6 days, and then adult occurred in about 10 days. A round of picking male fruit flies within 1 day after emergence was performed at a fixed time point each day for a total of 400 fruit flies, in which 20 fruit flies were placed in each tube labeled with the name, date, and number for a total of 20 tubes. Fruit flies were reared at 25° C., 50% humidity and 12 h light/12 h dark, the number of dead fruit flies in the tubes was counted and recorded every day, and the rearing tubes were changed every 3-4 days. The lifespan test for fruit flies in each group needed to be repeated three times independently and the recordings were entered into statistical software for analysis. The resulting survival curves were shown in FIG. A.

A tube-climbing test was then performed with the following steps:
1) 20 male fruit flies were collected by anesthetizing with CO2 and placed into a horizontally placed 250 mL clear glass measuring cylinder. A marker was made about 17.5 cm from the bottom.
2) The test was performed under ambient light with temperature and humidity of 22° C. and 50%, respectively. To avoid confusion of circadian rhythms, the test was always performed at the same time each day.
3) A sealing film was used to seal the top of the cylinder to prevent fruit flies from escaping.
4) The camera was placed on a tripod. The camera was aimed at the 190 ml line (i.e., at 17.5 cm) on the 250 ml graduated test tube.
(5) The fruit flies were moved onto the surface of the test tube bottom by repeatedly tapping 5-10 times, while the “Record” button on the camera was pressed to record the height at which the fruit flies climbed up to the marker (17.5 cm) within 2 min.
6) The test was repeated at least 5 times for each group of fruit flies, with an interval of at least 2 min between each test.
7) Analysis: the video was analyzed for each test, the total number of fruit flies that passed the marker was recorded every 10 s, and the percentage of fruit flies that were above the marker at each time point was derived.
8) The percentage of fruit flies were plotted for each time point, the performance at 120 s was analyzed, and the t-test was performed for both groups.

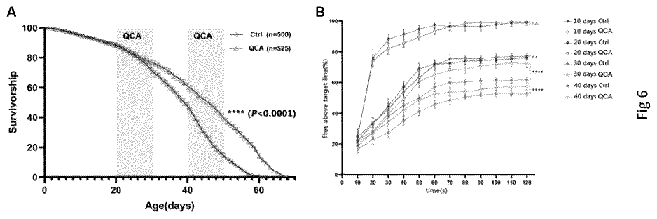

The results of the tube-climbing test were shown in FIG. 6B.

The survival curves showed that quinoline-4-carboxylic acid prolongs the lifespan of fruit flies, and the tube-climbing test showed that quinoline-4-carboxylic acid enhances their exercise capacity.

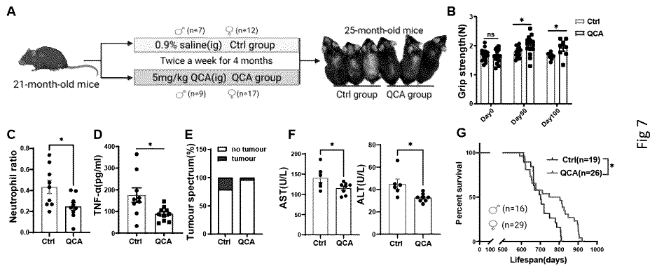

(7) 21 month-old C57BL6/J mice were randomly divided into control and QCA groups. 0.9% saline was given to the control group (n=7 for male mice, and n=12 for female mice); and 5 mg/kg QCA was given to the QCA group (n=9 for male mice, and n=13 for female mice) by consecutive gavage for 4 months. Pictures of the hairs of the mice in the Ctrl group and the QCA group were taken after 4 months, and the mice were observed for the coat status, dermatitis and other phenotypes, and the results were shown in FIG. 7A. The test mice were placed on the grip strength meter and the tail of the mice was gently pulled, so that the mice grasped the probe forcefully with the forelimbs and hindlimbs. The readings on the grip strength meter were recorded when the mice exerted the maximum force, the arm strength value in mice were obtained by averaging 3 repetitions of the measurements, and the results were shown in FIG. 7B. Blood was taken from the tail vein of mice 4 months after administration by gavage, the proportion of neutrophils (NEU) was measured by routine blood test, and the results were shown in FIG. 7C. Enzyme-linked immunosorbent assay (ELISA) was performed to detect the level of TNF-α in the serum of old mice in the QCA-treated and control groups, and the results were shown in FIG. 7D. The mice were examined periodically for the presence of spontaneous tumors, and the number of mice with spontaneous tumors in each group was recorded. The mouse tumor incidence in each group was obtained by dividing the number of mice with spontaneous tumors in each group by the total number of mice in each group, and the results were shown in FIG. 7E. Concentrations of alanine aminotransferase (ALT), aspartate aminotransferase (AST), and lactate dehydrogenase (LDH) in plasma were measured using a HITACHI 7600 Series (Japan) biochemical detector, and the results were shown in FIG. 7F. Survival times were recorded to make the survival curves of the mice in the Ctrl (n=19) and QCA (n=26) groups, and the results were shown in FIG. 7G.

The results showed that quinoline-4-carboxylic acid can reduce the symptoms such as poor coat and dermatitis in old mice; enhance the arm strength (reflecting the muscle strength of the mice), down-regulate the chronic inflammation and enzymatic indexes of liver injury in aged mice; and prolong the lifespan of the mice.

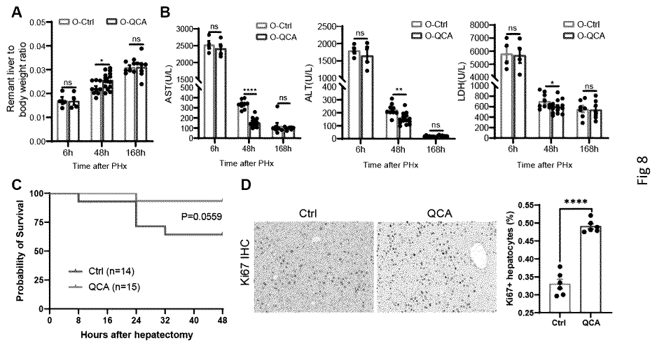

(8) Establishment of ⅔ partial hepatectomy model in old mice (⅔PHx): C57BL6/J mice older than 18 months were anesthetized with 2% isoflurane by inhalation and underwent a median abdominal incision. The left external and median lobes of the liver were resected separately with 4-0 silk sutures, and the associated vessels and bile ducts were ligated. Mice were injected intraperitoneally with 5 mg/kg QCA or an equal volume of DMSO 2 h before ⅔PHx surgery, and 24 h, 48 h and 72 h after ⅔PHx surgery, respectively. Mice were sacrificed at 6 h, 48 h and 168 h after surgery, respectively, and liver specimens and plasma were collected. The postoperative survival time of mice was recorded and used to plot survival curves. Mice were weighed in advance and livers were weighed after liver extraction so that the liver to body weight ratio could be calculated. Concentrations of alanine aminotransferase (ALT), aspartate aminotransferase (AST), and lactate dehydrogenase (LDH) in plasma were measured using a HITACHI 7600 Series (Japan) biochemical detector. Mouse liver samples collected at specific time points were fixed in 4% paraformaldehyde overnight and then embedded in paraffin. A 3 μm-thick paraffin section was made for subsequent Ki67 immunohistochemical staining, and the specific procedure was as follows: after subjecting to antigen repairing in EDTA (pH 8.0), the sections were incubated with a primary antibody overnight at 4° C., and then incubated with a secondary antibody at 37° C. for 1 h. Sections were stained with diaminobenzidine and hematoxylin, and then visualized and imaged with an optical microscope. The results were shown in FIG. 8.

The results showed that QCA supplementation does not significantly increase the regenerative liver to body weight ratio in mice at 6 h and 168 h after the hepatectomy, but it significantly increases the regenerative liver to body weight ratio in mice at 48 h after the hepatectomy (FIG. A). Also, QCA attenuates the enzymatic indexes of liver injury (FIG. B) and improves the survival rate of mice after hepatectomy (FIG. C). Ki67 immunohistochemical staining of liver tissue showed more Ki67-positive cells of liver tissue in the QCA group (FIG. D), indicating that QCA can promote liver regeneration.

The above-described examples are only to describe the preferred embodiment of the present disclosure, not to limit the scope of the present disclosure. On the premise of not departing from the spirit of the design according to the present disclosure, those skilled in the art can make various modifications and improvements to the technical solutions of the present disclosure, all of which should fall into the scope of protection determined by the claims of the present disclosure.

What is claimed is:

1. Use of quinoline-4-carboxylic acid in the preparation of an anti-aging product.

2. The use according to claim 1, wherein the anti-aging product is specifically a product for retarding the aging of mesenchymal stem cells.

3. The use according to claim 2, wherein the product for retarding the aging of mesenchymal stem cells is specifically a product for promoting the proliferation of mesenchymal stem cells.

4. The use according to claim 2, wherein the product for retarding the aging of mesenchymal stem cells is specifically a product for reducing the nuclear DNA damage of mesenchymal stem cells.

5. The use according to claim 1, wherein the anti-aging product is specifically a product for enhancing exercise capacity and muscle strength.

6. Use of quinoline-4-carboxylic acid in the preparation of a product for protecting liver.

7. Use of quinoline-4-carboxylic acid in the preparation of a product for promoting liver regeneration.

8. Use of quinoline-4-carboxylic acid in the preparation of a product for improving the survival rate after hepatectomy.

9. The use according to any one of claims 6 to 8, wherein the product for protecting liver, the product for promoting liver regeneration, and the product for improving the survival rate after hepatectomy are specifically a product for reducing liver inflammation.

10. The use according to any one of claims 6 to 8, wherein the product for protecting liver, the product for promoting liver regeneration, and the product for improving the survival rate after hepatectomy are specifically a product for reducing liver injury.
